(19) United States
(12) Patent Application Publication
(10) Pub. No.: US 2014/0296748 A1
(43) Pub. Date: Oct. 2, 2014

(54) MEASURING APPARATUS, METHOD OF DETERMINING MEASUREMENT REGION, AND PROGRAM

(71) Applicant: FUJITSU LIMITED, Kawasaki-shi (JP)

(72) Inventors: Hiroki ITAGAKI, Kawasaki (JP); Takayuki YAMAJI, Yokohama (JP)

(73) Assignee: FUJITSU LIMITED, Kawasaki-shi (JP)

(21) Appl. No.: 14/219,117

(22) Filed: Mar. 19, 2014

(30) Foreign Application Priority Data
Mar. 29, 2013 (JP) 2013-075025

(51) Int. Cl. A61B 5/00

(52) U.S. Cl. CPC A61B 5/4872
USPC 600/587

(57) ABSTRACT

A measuring apparatus for measuring predetermined information on a plurality of regions to be measured, the measuring apparatus includes: a first detection unit configured to detect a rotation mode of the measuring apparatus; and a determination unit configured to determine a region measured by the measuring apparatus on the basis of the rotation mode detected by the first detection unit in a movement process of the measuring apparatus from a predetermined position to any one of the regions.

### CROSS-REFERENCE TO RELATED APPLICATION

This application is based upon and claims the benefit of priority of the prior Japanese Patent Application No. 2013-075025, filed on Mar. 29, 2013, the entire contents of which are incorporated herein by reference.

### FIELD

The embodiments discussed herein are related to a measuring apparatus, a method of determining a measurement region, and a program.

### BACKGROUND

To date, there have been provided measuring apparatuses capable of deriving a body fat percentage by measuring subcutaneous fat thicknesses of individual regions, such as the backside of right and left arms, right and left abdominal regions (sides), the backside of right and left femoral regions, and the like. Among these measuring apparatuses, some of the measuring apparatuses are miniaturized. Accordingly, it is possible for a user to measure a body fat percentage of himself or herself by relatively easy operation.

Related-art techniques have been disclosed in Japanese Laid-open Patent Publication No. 2011-67344, and Japanese National Publication of International Patent Application No. 2011-523730.

However, in order to calculate a body fat percentage, each measured value has to be recorded as data being associated with corresponding body regions such as an upper arm, an abdominal region, a femoral region, and the like. Accordingly, a user has to input data on a region to be measured, by pressing a key and the like, every time the user measures a subcutaneous fat thickness of each of the regions.

### SUMMARY

According to an aspect of the invention, a measuring apparatus for measuring predetermined information on a plurality of regions to be measured, the measuring apparatus includes: a first detection unit configured to detect a rotation mode of the measuring apparatus; and a determination unit configured to determine a region measured by the measuring apparatus on the basis of the rotation mode detected by the first detection unit in a movement process of the measuring apparatus from a predetermined position to any one of the regions.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating an example of a hardware configuration of a mobile terminal according to a first embodiment;

FIGS. 2A and 2B are diagrams illustrating outer views of an example of the mobile terminal according to the first embodiment;

FIG. 3 is a diagram illustrating an example of a functional configuration of the mobile terminal according to the first embodiment;

FIG. 4 is a diagram illustrating a user's posture at the time of measuring a backside of a left upper arm;

FIGS. 5A and 5B are diagrams illustrating experimental data of detection values of an angular velocity sensor at the time of measuring right and left arms, respectively;

FIG. 6 is a diagram illustrating a relationship between detection values of an angular velocity sensor and a holding hand of a mobile terminal;

FIGS. 7A and 7B are diagrams illustrating experimental data of detection values of an acceleration sensor for each holding hand and for each measurement region;

FIG. 8 is a diagram illustrating relationships among a holding hand, detection values of an acceleration sensor, and a measurement region;

FIG. 9 is a flowchart for explaining an example of a processing procedure executed by a mobile terminal according to the first embodiment;

FIG. 10 is a flowchart for explaining an example of a processing procedure of measurement region determination processing according to the first embodiment;

FIG. 11 is a diagram illustrating an example of information stored in an angular-velocity related information storage unit according to a second embodiment;

FIG. 12 is a flowchart for explaining an example of a processing procedure executed by a mobile terminal according to a second embodiment;

FIG. 13 is a flowchart for explaining an example of a processing procedure of measurement region determination processing according to the second embodiment; and

FIG. 14 is a diagram illustrating a relationship between detection values of an acceleration sensor and a measurement region of a part of a face.

### DESCRIPTION OF EMBODIMENTS

In the following, descriptions will be given of embodiments of the present disclosure with reference to the drawings. In the present embodiment, a description will be given of an example of achieving an apparatus that measures a subcutaneous fat thickness and a body fat percentage using a mobile terminal, such as a smart phone, a mobile telephone, or the like. However, the present embodiment may be applied to a dedicated apparatus for measuring a subcutaneous fat thickness and a body fat percentage.

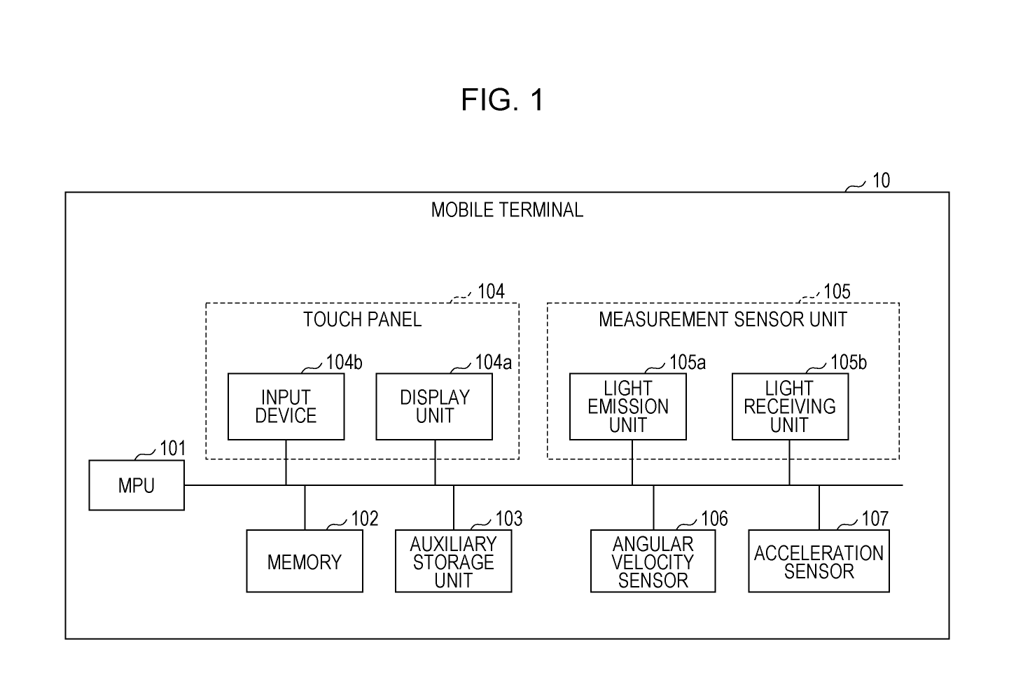

FIG. 1 is a diagram illustrating an example of a hardware configuration of a mobile terminal according to a first embodiment. In FIG. 1, a mobile terminal 10 includes an MPU 101, a memory 102, an auxiliary storage unit 103, a touch panel 104, a measurement sensor unit 105, an angular velocity sensor 106, and an acceleration sensor 107, and the like.

The auxiliary storage unit 103 stores a program installed in the mobile terminal 10, and so on. When an instruction to start the program is given, the program is read from the auxiliary storage unit 103, and is stored into the memory 102. The MPU 101 achieves functions related to the mobile terminal 10 in accordance with the program stored in the memory 102.

The touch panel 104 is an electronic component having both an input function and a display function, and displays information and receives input from a user, and so on. The touch panel 104 includes a display unit 104a and an input device 104b, and the like.

The display unit 104a is a liquid crystal display, or the like, and has a function of displaying on the touch panel 104. The input device 104b is an electronic component including a sensor that detects contact by a contact object on the display unit 104a. A method of detecting contact by a contact object may be any one of publicly known methods, such as an electrostatic method, a resistive film method, or an optical method, or the like. In this regard, a contact object means an object that touches the touch panel 104. Examples of these objects include a finger of a user, a dedicated pen or a general pen, and the like.

The measurement sensor unit 105 is hardware for measuring a subcutaneous fat thickness and a body fat percentage, and includes a light emitting unit 105a, a light receiving unit 105b, and the like. The light emitting unit 105a is, for example, a light emitting element, and emits light that enters skin, which is a biological surface. The light receiving unit 105b is, for example, a light receiving element, and detects the amount of received light that appears on the biological surface out of the incident light from the light emitting unit 105a. A subcutaneous fat thickness is calculated on the basis of the amount of received light. That is to say, a subcutaneous fat measurement technique of an optical method is used in the present embodiment. In this regard, the light emitting unit 105a and the light receiving unit 105b may not be a dedicated unit for measuring a subcutaneous fat thickness. For example, a light emitting element and a light receiving element for optical wireless communication using infrared rays in compliance with infrared data association (IrDA), which is generally included in a mobile telephone, a smart phone, and so on may be used.

The angular velocity sensor 106 is also referred to as a gyro sensor, and detects angular velocities in accordance with rotation movement of the mobile terminal 10. That is to say, the rotation mode of the mobile terminal 10 is detected by the angular velocity sensor 106. In this regard, for the angular velocity sensor 106, it is desirable to employ an angular velocity sensor of a biaxial type or more.

The acceleration sensor 107 detects acceleration of the mobile terminal 10 so as to detect a posture of the mobile terminal 10. In this regard, for the acceleration sensor 107, it is desirable to employ an acceleration sensor of a biaxial type or more.

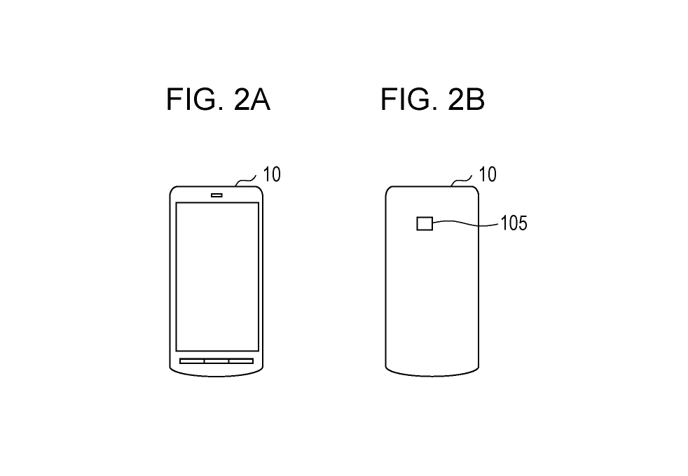

FIGS. 2A and 2B are diagrams illustrating outer views of an example of the mobile terminal according to the first embodiment. In FIGS. 2A and 2B, a same symbol is given to a same part as that in FIG. 1.

FIG. 2A illustrates a front view of the mobile terminal 10. FIG. 2B illustrates a back view of the mobile terminal 10. Also, the up and the down directions of the mobile terminal 10 individually match the up and the down directions in FIGS. 2A and 2B. The mobile terminal 10 according to the present embodiment is provided with the measurement sensor unit 105 at the upper part on the backside.

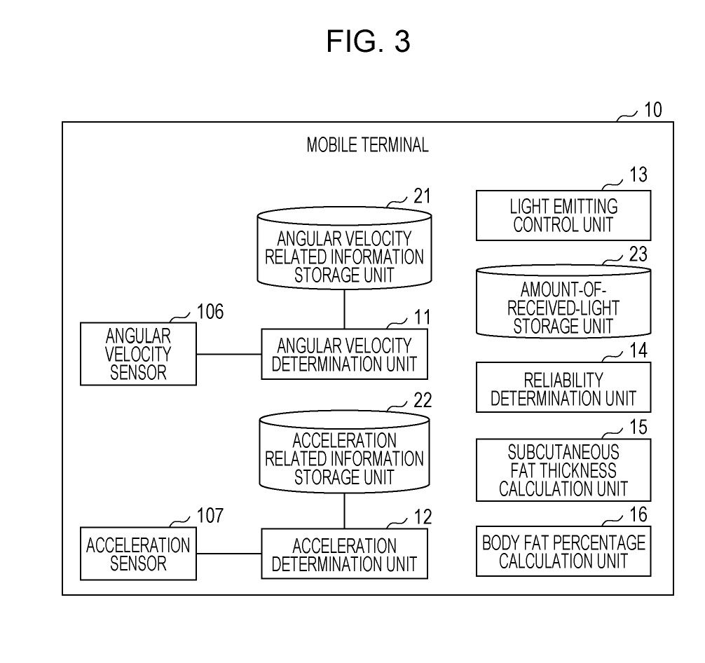

FIG. 3 is a diagram illustrating an example of a functional configuration of the mobile terminal according to the first embodiment. In FIG. 3, the mobile terminal 10 includes an angular velocity determination unit 11, an acceleration determination unit 12, a light emitting control unit 13, a reliability determination unit 14, a subcutaneous fat thickness calculation unit 15, and a body fat percentage calculation unit 16, and so on. Each of these units is achieved by the MPU 101 executing processing of the program installed in the mobile terminal 10. The mobile terminal 10 also includes an angular-velocity related information storage unit 21, an acceleration related information storage unit 22, and an amount-of-received light storage unit 23, and so on. It is possible to achieve each of these storage units using the auxiliary storage unit 103 or the memory 102, and so on.

The angular velocity determination unit 11 performs determination processing on the basis of angular velocities, which are detection values by the angular velocity sensor 106. In the first embodiment, the angular velocity determination unit 11 determines a holding hand of the mobile terminal 10 is determined on the basis of the angular velocities detected at the start time of a subcutaneous fat thickness. In the present embodiment, a “holding hand” means either a right hand or a left hand that holds the mobile terminal 10. Accordingly, the determination of a holding hand of the mobile terminal 10 means a determination of whether a right hand or a left hand holds the mobile terminal 10.

In the first embodiment, out of the backside of the right or the left upper arm, the right or the left abdominal region (side), and the backside of the right or left femoral region, the backside of either the right or the left upper arm is assumed to be the first region to be measured (hereinafter, a region to be measured is referred to as a “measurement region”). The angular velocity determination unit 11 determines a holding hand on the basis of the angular velocities detected by the angular velocity sensor 106 in the movement process period from input of an measurement instruction by a user into the mobile terminal 10 to putting the measurement sensor unit 105 to the backside of either the right or the left upper arm. That is to say, the rotation mode of the mobile terminal 10 is different between the case where the user holds the mobile terminal 10 by a right hand, and the case of holding by a left hand, which is caused by the difference in a natural rotation direction of a wrist. In the present embodiment, a determination of a holding hand is made by focusing attention on such a phenomenon.

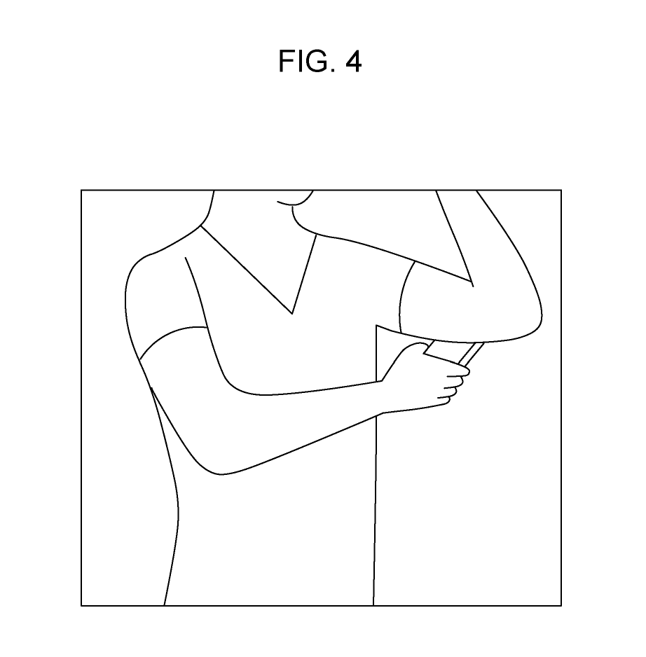

For example, when a user measures the backside of the left upper arm, the user assumes a posture as illustrated in FIG. 4. FIG. 4 is a diagram illustrating a user's posture at the time of measuring the backside of a left upper arm. As illustrated in FIG. 4, in order to assume a posture that is easy for measuring the backside of a left upper arm, the user raises the left upper arm. The user puts the backside of the mobile terminal 10 to the backside of the raised left upper arm so as to measure a subcutaneous fat thickness of the backside of the left upper arm. A determination is made that a holding hand is a right hand on the basis of the angular velocities detected in a changing process from a state in which the front side of the mobile terminal 10 is substantially facing the face of the user (hereinafter, referred to as a “reference state”) to the state of the mobile terminal 10 as illustrated in FIG. 4. In this regard, in the case where a right upper arm becomes the measurement region, the measurement is also carried out in the same posture except that the posture becomes symmetrical.

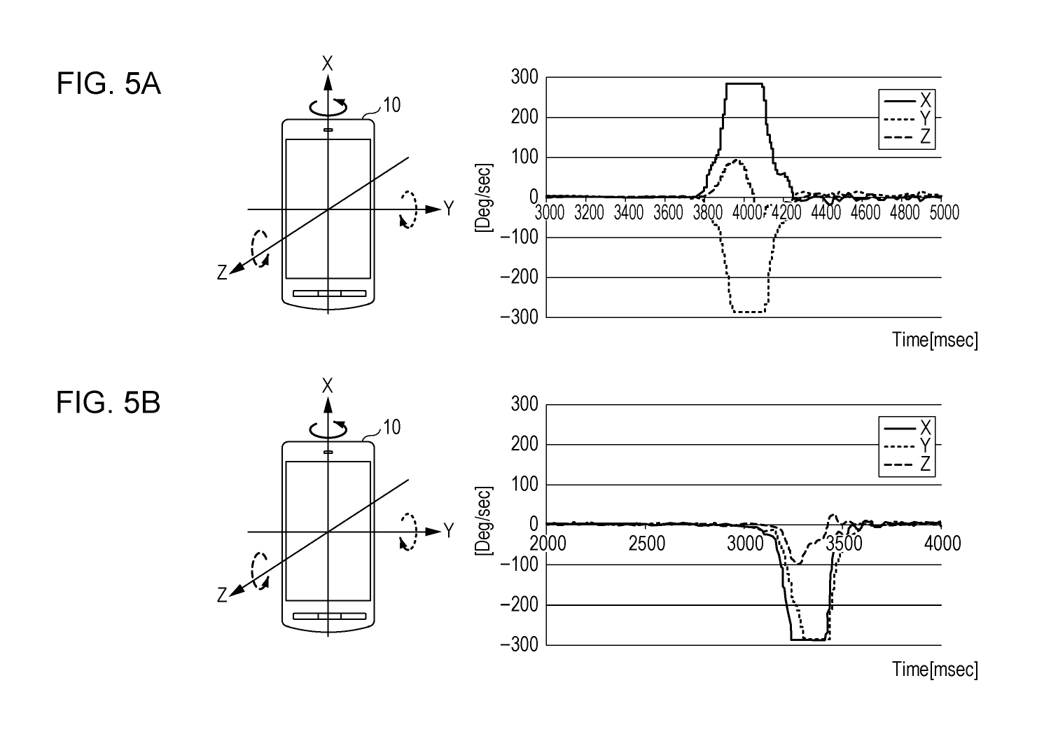

FIGS. 5A and 5B illustrate experimental data of the detection values by the angular velocity sensor 106 at the time of measuring the backside of a right arm and a left arm, respectively using the mobile terminal 10 in the posture as illustrated in FIG. 4.

FIGS. 5A and 5B are diagrams illustrating experimental data of the detection values of an angular velocity sensor at the time of measuring right and left arms, respectively.

On the left side in FIGS. 5A and 5B, the X-axis, the Y-axis, and the Z-axis of the angular velocity sensor 106 of the mobile terminal 10, which was used for the experiment, and the positive rotation directions of the individual axes are illustrated. The angular velocity sensor 106 assumes that the up-and-down direction of the mobile terminal 10 is the X-axis, the left-and-right direction is the Y-axis, and the front-and-back direction is the Z-axis. Also, the angular velocity sensor 106 assumes that the counterclockwise direction about the X-axis as seen in the top view of the mobile terminal 10 is the positive rotation direction of the X-axis. Also, the angular velocity sensor 106 assumes that the clockwise direction about the Y-axis as seen in the right side view of the mobile terminal 10 is the positive rotation direction of the Y-axis. Also, the angular velocity sensor 106 assumes that the counterclockwise direction about the Z-axis as seen in the front view of the mobile terminal 10 is the positive rotation direction of the Z-axis.

The graphs on the right side of FIGS. 5A and 5B denote the detection values of the angular velocity sensor 106 at the time of the experiments. The graphs in FIG. 5A are the angular velocities detected by the angular velocity sensor 106 in the process in which the mobile terminal 10 held by a left hand is moved from the reference state to a state of putting the measurement sensor unit 105 to the backside of the right upper arm. The graphs in FIG. 5B are the angular velocities detected by the angular velocity sensor 106 in the process in which the mobile terminal 10 held by a right hand is moved from the reference state to a state of putting the measurement sensor unit 105 to the backside of the left upper arm.

As illustrated in FIG. 5A, in the case where the backside of the right upper arm is measured with the mobile terminal 10 held by a left hand, a rotation is detected in the plus direction (angular velocity) with respect to the X-axis is detected, and the rotation is detected in the negative direction (angular velocity) with respect to the Y-axis.

On the other hand, as illustrated in FIG. 5B, in the case where the backside of the left upper arm is measured with the mobile terminal 10 held by a right hand, a rotation is detected in the negative direction (angular velocity) with respect to the X-axis is detected, and the rotation is detected in the negative direction (angular velocity) with respect to the Y-axis.

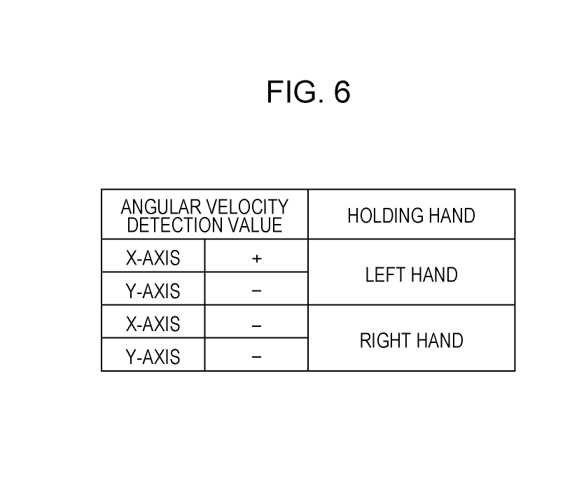

The above relationships are summarized as illustrated in FIG. 6. FIG. 6 is a diagram illustrating a relationship between the detection values of the angular velocity sensor and the holding hand of the mobile terminal.

As illustrated in FIG. 6, in the case of the mobile terminal 10 according to the present embodiment, at the time of measuring an upper arm, if a positive angular velocity on the X-axis is detected, and a negative angular velocity on the Y-axis is detected, there is a high possibility that a left hand is the holding hand. On the other hand, if a negative angular velocity on the X-axis is detected, and a negative angular velocity on the Y-axis is detected, there is a high possibility that a right hand is the holding hand.

The angular velocity determination unit 11 determines a holding hand of the mobile terminal 10 on the basis of the information illustrated in FIG. 6. Also, the angular-velocity related information storage unit 21 stores the information illustrated in FIG. 6. In this regard, the relationship between the angular velocities and the holding hand may differ depending on the position of the measurement sensor unit 105 on the mobile terminal 10, the definitions of each axis of the angular velocity sensor 106, a positive rotation direction, and so on. Accordingly, for example, a relationship between the angular velocities and the holding hand may be defined for each model of the mobile terminal 10, and so on.

In this regard, when the mobile terminal 10 is held by the right hand, the backside of the left upper arm, the right abdominal region, and the backside of the right femoral region are assumed to be measurement regions. On the other hand, when the mobile terminal 10 is held by the left hand, the backside of the right upper arm, the left abdominal region, and the backside of the left femoral region are assumed to be measurement regions. This is because when a user measures a subcutaneous fat thickness of each region of his or her own, it is thought that such methods of measuring are natural operations.

Also, after completion of measurement on all the measurement regions by one hand, it is assumed that measurement on all the measurement regions by the other hand is carried out. For example, when measurement is carried out with the mobile terminal 10 held by a right hand first, the backside of the left upper arm is determined to be a measurement region first. After that, the right abdominal region and the backside of the right femoral region are determined to be the measurement regions in random order. Next, the mobile terminal 10 is held by a left hand, and the backside of the right upper arm is determined to be a measurement region. After that, the left abdominal region and the backside of the left femoral region are determined to be the measurement regions in random order.

Referring back to FIG. 3, the acceleration determination unit 12 performs determination processing on the basis of the detection values by the acceleration sensor 107. In the first embodiment, the acceleration determination unit 12 determines a measurement region on the basis of the determination result by the angular velocity determination unit 11, and the detection values by the acceleration sensor 107. That is to say, the acceleration determination unit 12 determines a measurement region on the basis of the information indicating whether the mobile terminal 10 is held by a left hand or a right hand, and the posture of the mobile terminal 10. Regarding the mobile terminal 10 of the present embodiment, experimental data of the detection values detected by the acceleration sensor 107 for each holding hand and for each measurement region are as follows. In this regard, hereinafter, for the upper arm and the femoral region, the “backside” is omitted.

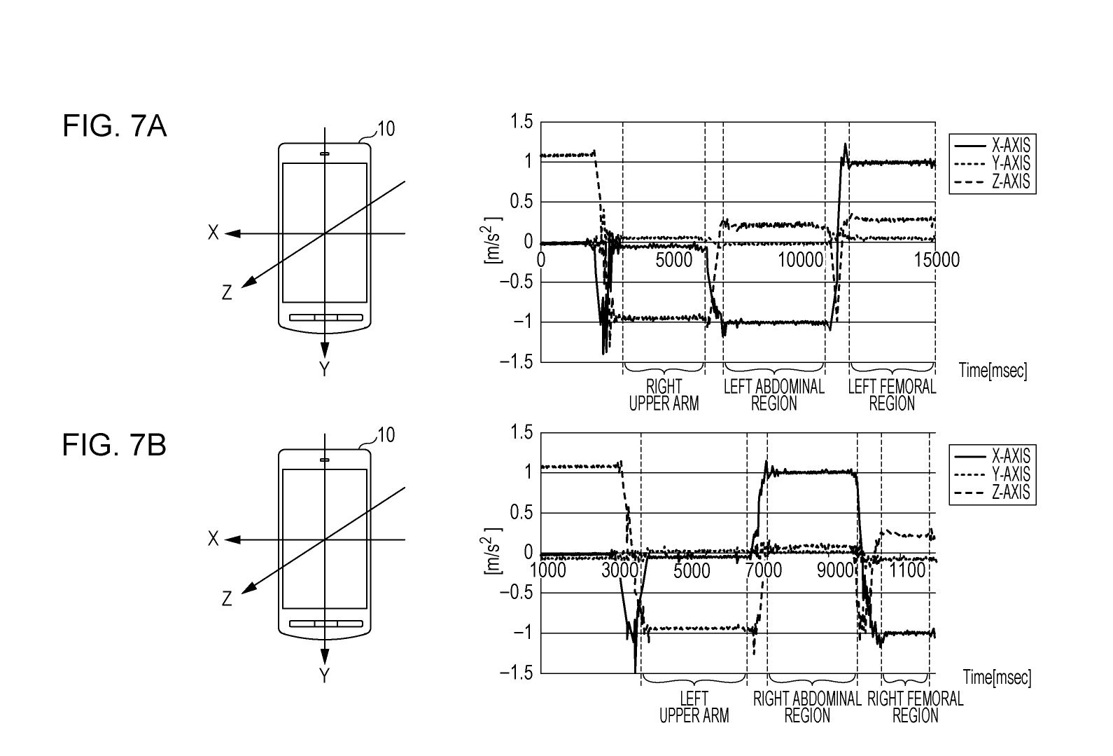

FIGS. 7A and 7B are diagrams illustrating experimental data of the detection values of the acceleration sensor for each holding hand and for each measurement region.

In FIGS. 7A and 7B, the mobile terminal 10 on the left side illustrates the X-axis, the Y-axis, and the Z-axis, and the positive directions of acceleration on the individual axes. That is to say, the direction indicated by an arrow in each axis is a positive direction. In this regard, at the time of measurement, the mobile terminal 10 is substantially in a stationary state, and thus the detected acceleration is the gravity acceleration. Accordingly, for example, in a state in which the front face is facing downward, a positive acceleration is detected on the Z-axis.

The graphs on the right side of FIG. 7A illustrate detection values of the acceleration sensor 107 at the time of measuring each region when the left hand is the holding hand. The graphs in FIG. 7B illustrate the detection values of the acceleration sensor 107 at the time of measuring each region when the right hand is the holding hand.

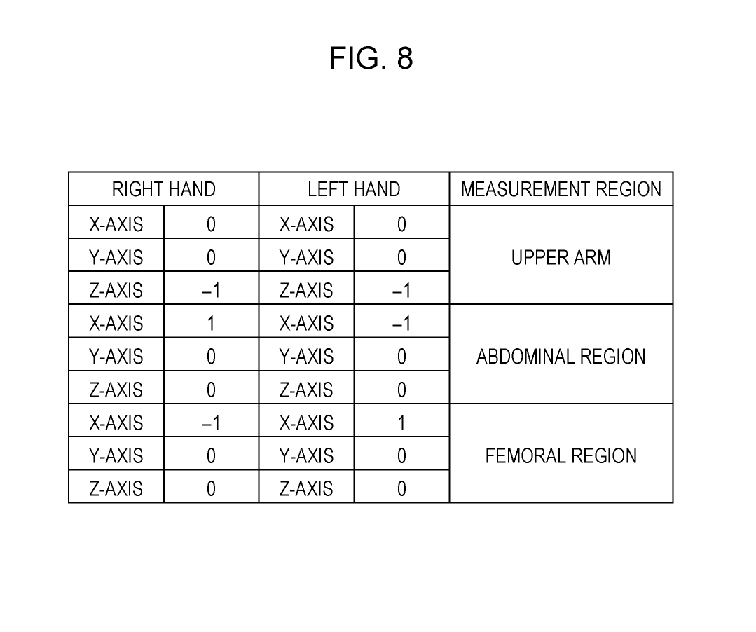

The summary of the contents of the graphs in FIGS. 7A and 7B becomes as illustrated in FIG. 8. FIG. 8 is a diagram illustrating relationships among a holding hand, detection values of the acceleration sensor, and a measurement region.

In FIG. 8, when the holding hand is a right hand, if the acceleration on the X-axis is about 0, the acceleration on the Y-axis is about 0, and the acceleration on the Z-axis is about −1, it is indicated that the measurement region is an upper arm (left upper arm). Also, when the holding hand is a right hand, if the acceleration on the X-axis is about 1, the acceleration on the Y-axis is about 0, and the acceleration on the Z-axis is about 0, it is indicated that the measurement region is an abdominal region (right abdominal region). Further, when the holding hand is a right hand, if the acceleration on the X-axis is about −1, the acceleration on the Y-axis is about 0, and the acceleration on the Z-axis is about 0, it is indicated that the measurement region is a femoral region (right femoral region).

Also, when the holding hand is a left hand, if the acceleration on the X-axis is about 0, the acceleration on the Y-axis is about 0, and the acceleration on the Z-axis is about −1, it is indicated that the measurement region is an upper arm (right upper arm). Also, when the holding hand is a left hand, if the acceleration on the X-axis is about −1, the acceleration on the Y-axis is about 0, and the acceleration on the Z-axis is about 0, it is indicated that the measurement region is an abdominal region (left abdominal region). Further, when the holding hand is a left hand, if the acceleration on the X-axis is about 1, the acceleration on the Y-axis is about 0, and the acceleration on the Z-axis is about 0, it is indicated that the measurement region is a femoral region (left femoral region).

The acceleration determination unit 12 determines the measurement region on the basis of the information indicated in FIG. 8. Also, the acceleration related information storage unit 22 stores the information indicated in FIG. 8. In this regard, a relationship among a holding hand, detection values of the acceleration sensor 107, and a measurement region may be different depending on the position of the measurement sensor unit 105 in the mobile terminal 10, the definitions of the individual axes, the positive directions of the acceleration sensor 107, and so on. Accordingly, for example, a relationship among a holding hand, detection values of the acceleration sensor 107, and a measurement region may be defined for each model of the mobile terminal 10, and so on.

Referring back to FIG. 3, the light emitting control unit 13 controls light emission by the light emitting unit 105a. The amount-of-received light storage unit 23 stores values indicating the amount of received light by the light receiving unit 105b. The reliability determination unit 14 determines the reliability of the values of the amount of received light, which has been stored in the amount-of-received light storage unit 23. For example, if outside light is mixed in the amount of received light, the value of the amount of received light becomes significantly high. Accordingly, in such a case, the reliability determination unit 14 determines that the reliability of the value of the amount of received light is low. The subcutaneous fat thickness calculation unit 15 calculates a subcutaneous fat thickness on the basis of the value of the amount of received light that has been stored in the amount-of-received light storage unit 23. For the calculation of a subcutaneous fat thickness on the basis of the amount of received light, a publicly known technique ought to be used. For example, information indicating a relationship between the amount of received light and the subcutaneous fat thickness may be stored in advance, and a subcutaneous fat thickness may be calculated on the basis of the information. Also, the information may be selected from a plurality of candidates in accordance with a user's age, and so on. The body fat percentage calculation unit 16 calculated a body fat percentage on the basis of a subcutaneous fat thickness of each region. For a method of calculating a body fat percentage on the basis of a subcutaneous fat thickness, a publicly known technique may be used.

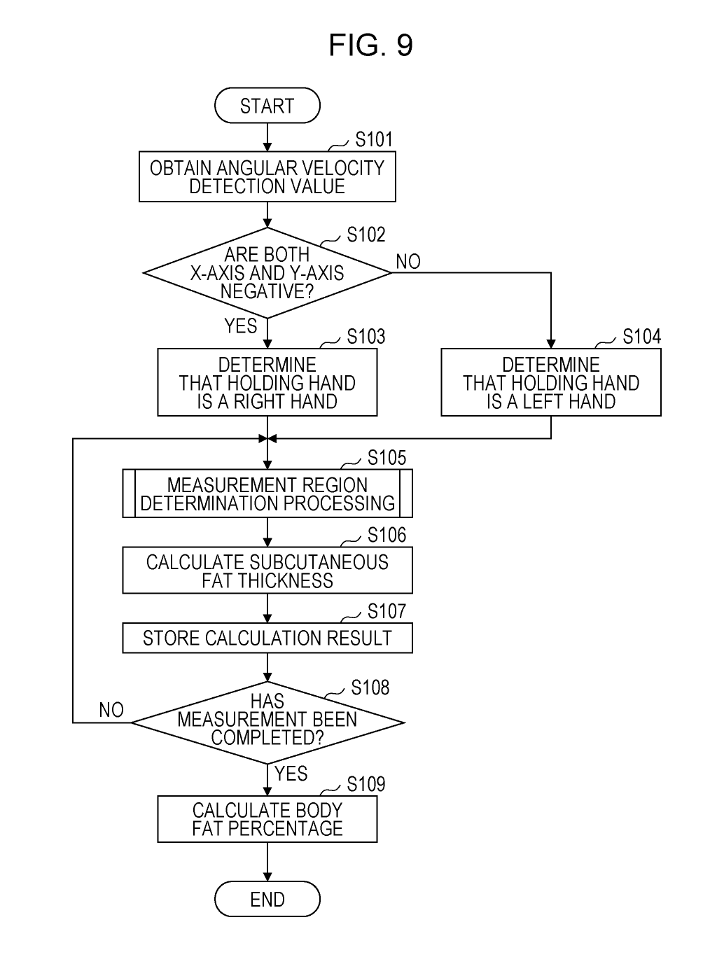

In the following, a description will be given of a processing procedure executed by the mobile terminal 10 in the first embodiment. FIG. 9 is a flowchart for explaining an example of a processing procedure executed by the mobile terminal according to the first embodiment. The processing in FIG. 9 is started, for example by a user inputting a measurement instruction through the touch panel 104, or the like.

In step S101, the angular velocity determination unit 11 obtains the detection value of the angular velocities from the angular velocity sensor 106. That is to say, time-series detection values of the angular velocities are obtained in the process in which the mobile terminal 10 moves from an initial state to a measurement position of a subcutaneous fat thickness of either a left or a right upper arm. As a result, the detection values as illustrated in FIG. 5A or FIG. 5B are obtained.

Next, the angular velocity determination unit 11 compares the obtained measurement value with the information stored in the angular-velocity related information storage unit 21 (FIG. 6) so as to determine a holding hand. In the present embodiment, a determination is made of whether an angular velocity not higher than a negative threshold value has been detected in the X-axis direction, and an angular velocity not higher than a negative threshold value has been detected in the Y-axis direction on the basis of the information illustrated in FIG. 6 (S102). If an angular velocity not higher than the negative threshold value has been detected in the X-axis direction, and an angular velocity not higher than the negative threshold value has been detected in the Y-axis direction (Yes in S102), the angular velocity determination unit 11 determines that the holding hand is a right hand (S103). In the other case (No in S102), the angular velocity determination unit 11 determines that the holding hand is a left hand (S104). The determination result by the angular velocity determination unit 11 is stored in the memory 102, for example.

Next, when the measurement sensor unit 105 is put to a measurement region, the acceleration determination unit 12 performs measurement region determination processing (S105). A detailed description will be given later of a processing procedure of the measurement region determination processing. A determination result of the measurement region, which is the processing result of the measurement region determination processing is stored in the memory 102, for example. In this regard, the fact that the measurement sensor unit 105 has been put to a measurement region may be determined by the fact that the amount of received light by the light receiving unit 105b becomes small, for example.

Next, the mobile terminal 10 measures a subcutaneous fat thickness (S106). Specifically, the light emitting unit 105a emits light under the control of the light emitting control unit 13. The light receiving unit 105b detects the amount of received light that appears on a biological surface, which is a skin of the user, and stores the value indicating the amount of received light into the amount-of-received light storage unit 23. The reliability determination unit 14 determines the reliability of the value of the amount of received light stored in the amount-of-received light storage unit 23. If the reliability determination unit 14 has determined that there is reliability in the value of the amount of received light, the subcutaneous fat thickness calculation unit 15 calculates a subcutaneous fat thickness on the basis of the value of the amount of received light.

Next, the subcutaneous fat thickness calculation unit 15 associates the calculation result of the subcutaneous fat thickness with the determination result of the measurement region in step S105, and stores the calculation result and the determination result in the memory 102 or the auxiliary storage unit 103, for example (S107). That is to say, the information indicating the measurement region and the calculation result of the subcutaneous fat thickness are stored in association with each other.

The processing in steps S105 to S107 is executed for each region to be measured. In this regard, the mobile terminal 10 may give an instruction on the next measurement region to the user using an audio guidance, or the like. For example, a message stating “Please measure the right abdominal region” may be output. Even in this case, the user might make a mistake in a measurement region, and thus the execution of the measurement region determination processing is meaningful. Also, in order to avoid removing the mobile terminal 10 by the user from the region before completion of the measurement of the subcutaneous fat thickness of each region, the mobile terminal 10 may make a confirmation sound indicating completion of the measurement, and so on every time the measurement of each region is complete.

When the measurement of a subcutaneous fat thickness of all the regions to be measured is complete (Yes in S108), the body fat percentage calculation unit 16 calculates a body fat percentage on the basis of the subcutaneous fat thickness of each region, which is stored in the memory 102 or the auxiliary storage unit 103 (S109). The calculation result of the subcutaneous fat thickness for each region, and the calculation result of the body fat percentage are displayed on the display unit 104a of the touch panel 104, for example. In this regard, the completion of the measurement of the subcutaneous fat thicknesses of all the regions may be determined automatically by the mobile terminal 10, or may be determined on the basis of input by the user.

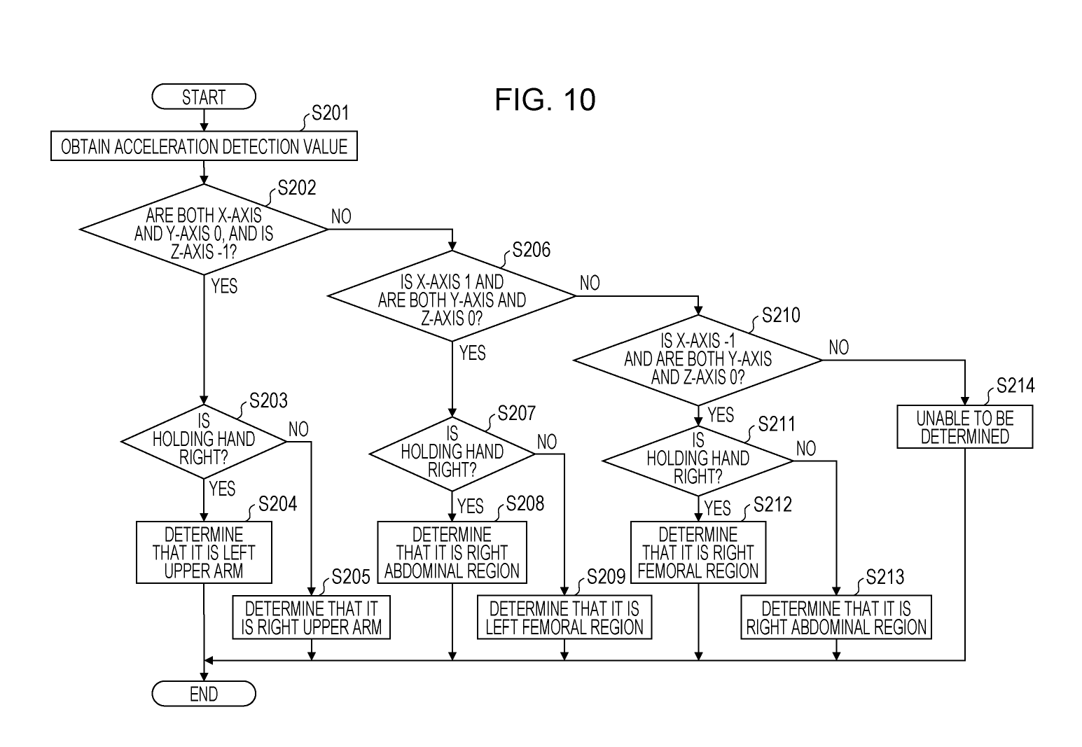

Next, a description will be given of details of step S105. FIG. 10 is a flowchart for explaining an example of a processing procedure of the measurement region determination processing according to the first embodiment.

In step S201, the acceleration determination unit 12 obtains the detection values of acceleration from the acceleration sensor 107. Next, the acceleration determination unit 12 determines a measurement region on the basis of the information stored in the acceleration related information storage unit 22 (FIG. 8).

For example, in step S202, the acceleration determination unit 12 determines whether the detection value on the X-axis is 0, the detection value on the Y-axis is 0, and the detection value on the Z-axis is −1. In this regard, each of the detection values may not be strictly 0, 1, or −1. For example, if an acceleration value not higher than a negative threshold value is detected, the acceleration determination unit 12 may round off the detection value to −1. If an acceleration value not lower than a positive threshold value is detected, the acceleration determination unit 12 may round off the detection value to 1. In other cases, the acceleration determination unit 12 may round off the detection value to 0.

If the detection values of the individual axes match the compared corresponding values, respectively (Yes in S202), the acceleration determination unit 12 determines whether the holding hand is a right hand or not (S203). The information indicating the holding hand is stored in the memory 102 in step S103 or S104 in FIG. 9, for example.

If the holding hand is a right hand (Yes in S203), the acceleration determination unit 12 determines that the left upper arm is the measurement region (S204). If the holding hand is a left hand (No in S203), the acceleration determination unit 12 determines that the right upper arm is the measurement region (S205).

On the other hand, if No in step S202, the acceleration determination unit 12 determines whether the detection value on the X-axis is 1, the detection value on the Y-axis is 0, and the detection value on the Z-axis is 0, for example (S206). If the detection values of the individual axes match the compared corresponding values, respectively (Yes in S206), the acceleration determination unit 12 determines whether the holding hand is a right hand or not (S207). If the holding hand is a right hand (Yes in S207), the acceleration determination unit 12 determines that the right abdominal region is the measurement region (S208). If the holding hand is a left hand (No in S203), the acceleration determination unit 12 determines that the left femoral region is the measurement region (S209).

On the other hand, if No in step S206, the acceleration determination unit 12 determines whether the detection value on the X-axis is −1, the detection value on the Y-axis is 0, and the detection value on the Z-axis is 0, for example (S210). If the detection values of the individual axes match the compared corresponding values, respectively (Yes in S210), the acceleration determination unit 12 determines whether the holding hand is a right hand or not (S211). If the holding hand is a right hand (Yes in S211), the acceleration determination unit 12 determines that the right femoral region is the measurement region (S212). If the holding hand is a left hand (No in S203), the acceleration determination unit 12 determines that the left abdominal region is the measurement region (S213).

As described above, by the first embodiment, it is possible for the mobile terminal 10 to automatically determine a measurement region. Accordingly, it is possible for the user to reduce the work of recording a measurement region. As a result, it is possible to shorten time for continuously measuring a subcutaneous fat thickness of each region, for example.

In this regard, in the present embodiment, an example in which a holding hand is also automatically determined has been illustrated. However, whether the holding hand is a right hand or a left hand may be input by the user. The number of input of the holding hand is two at the most, and thus it is thought that user's operation workload is small compared with input operation of each measurement region.

Also, the first measurement region for determining the holding hand may be other than an upper arm. In this case, the information illustrated in FIG. 6 ought to be changed to meet the first measurement region.

Also, for a measuring apparatus that is operated with a fixed holding hand, namely either a right or left hand, the determination processing of the holding hand may be omitted.

Next, a description will be given of a second embodiment. In the second embodiment, a description will be given of points that are different from those in the first embodiment. Accordingly, the points that are not mentioned in particular may be the same as those in the first embodiment.

In the first embodiment, a description has been given of an example in which a holding hand of the mobile terminal 10 is determined on the basis of the mode of rotation of the mobile terminal 10 at the time of measuring a subcutaneous fat thickness of either a right or a left upper arm, and a measurement region is determined on the basis of the determination result of the holding hand and the detection values of the acceleration sensor 107. A method of determining a measurement region on the basis of such a procedure is based on the assumption that the first measurement region is fixed. That is to say, in the first embodiment, it is assumed that the first measurement region is either a right or a left upper arm.

In the second embodiment, a description will be given of an example that allows improving the degree of freedom of a first measurement region. That is to say, in the second embodiment, a description will be given of an example that allows determination of each measurement region without fixing the first measurement region.

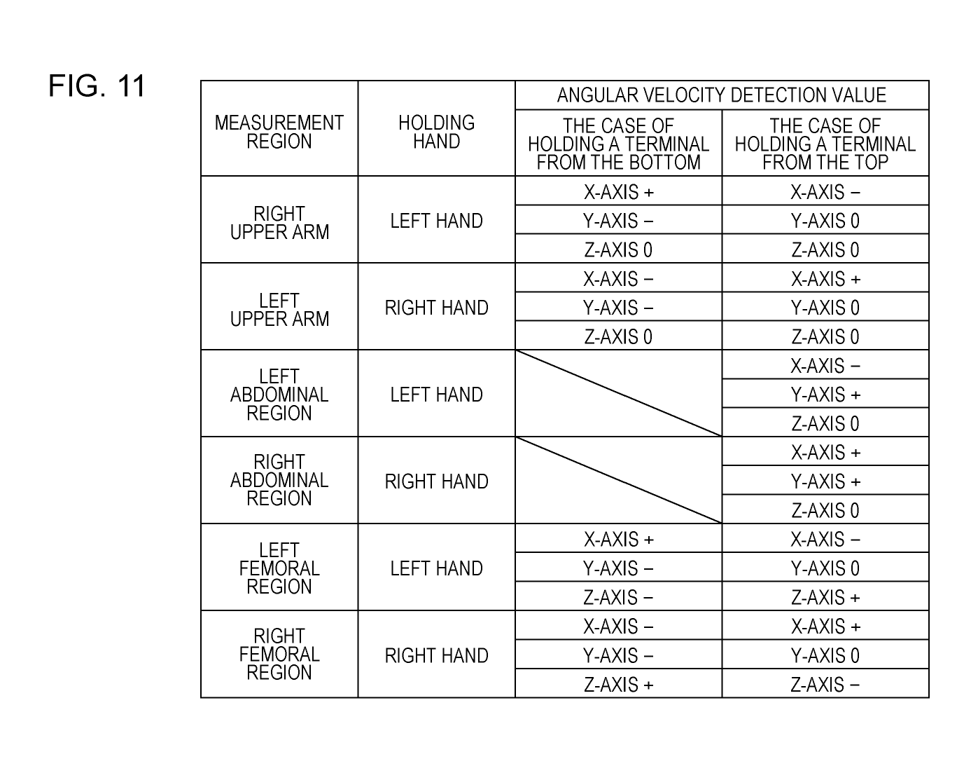

FIG. 11 is a diagram illustrating an example of information stored in an angular-velocity related information storage unit according to a second embodiment. As illustrated in FIG. 11, an angular-velocity related information storage unit 21 according to the second embodiment stores corresponding relationships among the measurement region, the holding hand, the detection values of the angular velocities in the case of holding the mobile terminal 10 from the bottom or from the top. In this regard, the detection values of the angular velocities corresponding to each measurement region, which are illustrated in FIG. 11, are angular velocities that are detected in the process of individually moving the mobile terminal 10 from the reference state to each measurement region. Also, the case of holding the mobile terminal 10 from the bottom means the case of holding the mobile terminal 10 such that the backside of the mobile terminal 10 faces a palm of the user. The case of holding the mobile terminal 10 from the top means the case of holding the mobile terminal 10 such that the surface of the mobile terminal 10 is opposed to the palm of the user. In this regard, the separation of the cases where the mobile terminal 10 is held from the bottom or from the top may be taken into consideration in the first embodiment.

By the information illustrated in FIG. 11, in the case of holding the mobile terminal 10 from the bottom, if the detection values of the angular velocities in the X-axis direction, in the Y-axis direction, and in the Z-axis direction are positive, negative, and about 0, respectively, it is understood that there is a high possibility that the holding hand is a left hand, and the measurement region is a right upper arm.

In this regard, in the second embodiment, the information stored in the acceleration related information storage unit 22 is as illustrated is FIG. 8 in the same manner as the first embodiment.

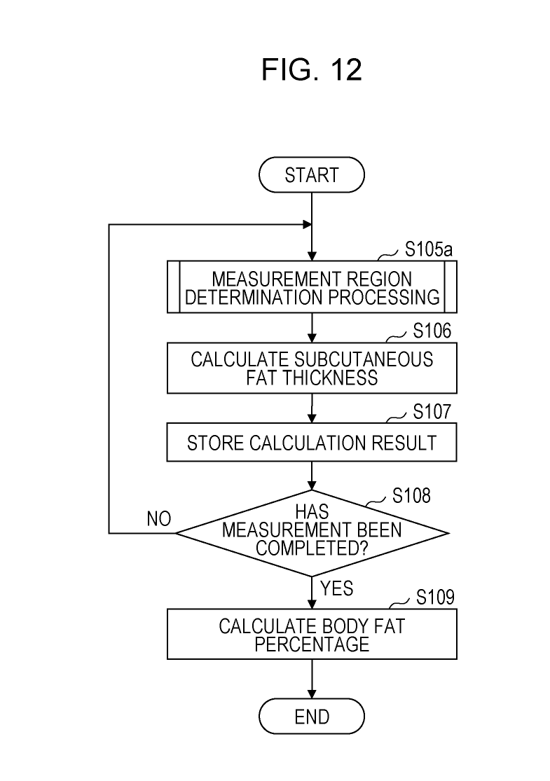

In the following, a description will be given of a processing procedure executed by the mobile terminal 10 in the second embodiment. FIG. 12 is a flowchart illustrating an example of a processing procedure executed by the mobile terminal according to the second embodiment. In FIG. 12, the same step number is given to the same step as that in FIG. 10, and the description thereof will be omitted.

In FIG. 12, steps S101 to S104 are removed. Also, step S105 is replaced by step S105a. The other steps are the same as those in FIG. 9.

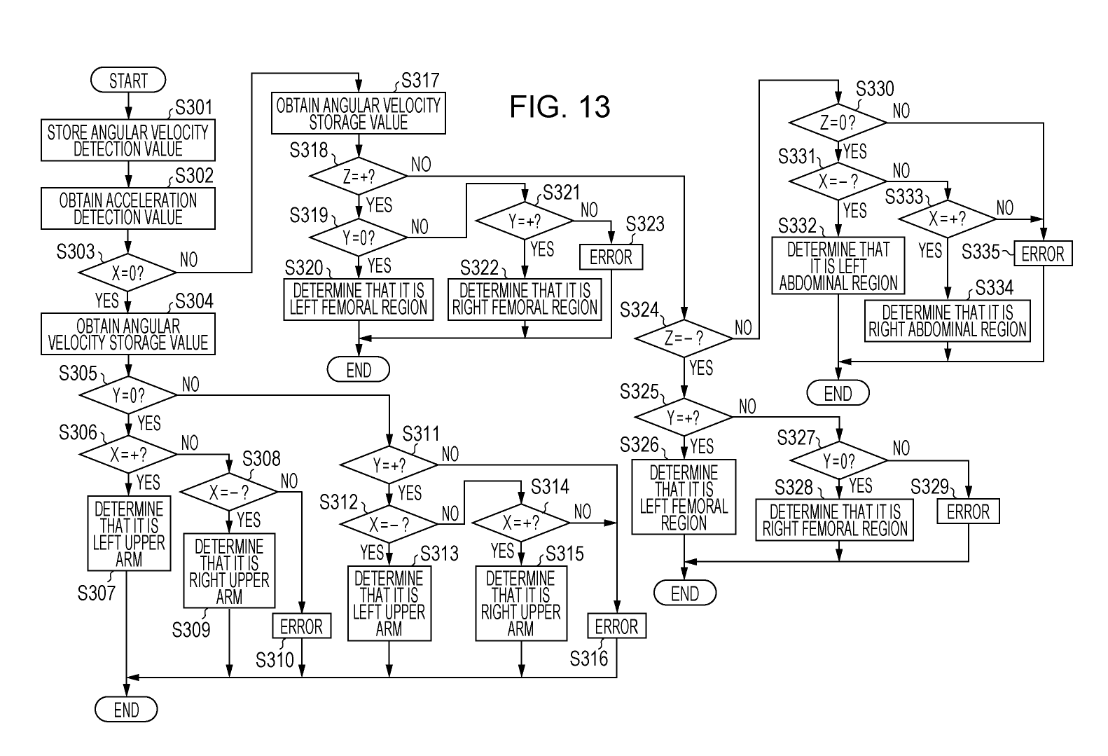

Next, a detailed description will be given of step S105a. FIG. 13 is a flowchart illustrating an example of a processing procedure of the measurement region determination processing according to the second embodiment.

In step S301, angular velocity determination unit 11 obtains detection values of the angular velocities from the angular velocity sensor 106, and stores the obtained detection values into the memory 102, for example. For example, if an angular velocity not lower than a positive threshold value is detected, “+” is stored, if an angular velocity not higher than a negative threshold value, “−” is stored, and in the other cases, “0” is stored individually into the X-axis, the Y-axis, and the Z-axis fields. In this regard, step S301 is executed in the process in which the mobile terminal 10 moves from the reference state to any one of the measurement regions.

The subsequent step S302 and after that are executed after the mobile terminal 10 is put to any one of the measurement regions. In step S302, the acceleration determination unit 12 obtains the detection values of acceleration from the acceleration sensor 107. In this regard, the detection values of acceleration may be rounded off to 0, 1, or −1 in the same manner as the first embodiment. Next, the acceleration determination unit 12 determines whether the detection value of acceleration in the X-axis direction is 0 or not (S303). If the detection value of acceleration in the X-axis direction is 0 (Yes in S303), the angular velocity determination unit 11 obtains the angular velocity value (hereinafter, referred to as an “angular velocity storage value”) that has been detected in step S301 and stored in the memory 102 (S304).

Next, the angular velocity determination unit 11 determines whether the angular velocity storage value in the Y-axis direction is 0 or not (S305). If the angular velocity storage value in the Y-axis direction is 0 (Yes in S305), the angular velocity determination unit 11 determines whether the angular velocity storage value in the X-axis direction is “+” or not (S306). If the angular velocity storage value in the X-axis direction is “+” (Yes in S306), the angular velocity determination unit 11 determines that the left upper arm is the measurement region (S307). That is to say, the processing of step S307 is executed in the case where the acceleration in the X-axis direction is 0, and the angular velocity storage values are 0 in the Y-axis direction and “+” in the X-axis direction. The state in which the acceleration is 0 is a state detected when the measurement region is either a right or a left upper arm with reference to FIG. 8. Also, when the measurement region is either a right or a left upper arm, the state in which the angular velocity storage values are 0 in the Y-axis direction and “+” in the X-axis direction, respectively is a state detected when the left upper arm is measured with the mobile terminal 10 held by a right hand from the top with reference to FIG. 11. Accordingly, in step S307, the angular velocity determination unit 11 determines that the left upper arm is the measurement region.

On the other hand, if the angular velocity storage value in the X-axis direction is not “+” (No in S306), the angular velocity determination unit 11 determines whether the angular velocity storage value in the X-axis direction is “−” or not (S308). If the angular velocity storage value in the X-axis direction is “−” (Yes in S308), the angular velocity determination unit 11 determines that a right upper arm is the measurement region (S309). That is to say, the processing of step S309 is executed in the case where the acceleration in the X-axis direction is 0, and the angular velocity storage value in the Y-axis direction is 0, and the angular velocity storage value in the X-axis direction is “−”. The state in which the acceleration is 0 is a state detected when the measurement region is either a right or a left upper arm with reference to FIG. 8. Also, when the measurement region is either a right or a left upper arm, the state in which the angular velocity storage values are 0 in the Y-axis direction and “−” in the X-axis direction, respectively is a state detected when the right upper arm is measured with the mobile terminal 10 held by a left hand from the top with reference to FIG. 11. Accordingly, in step S309, the angular velocity determination unit 11 determines that the right upper arm is the measurement region.

In step S308, if the angular velocity storage value in the X-axis direction is not “−” (No in S308), the determination processing of a measurement region becomes an error. An error means being incapable of determination.

Also, in step S305, if the angular velocity storage value in the Y-axis direction is not 0 (No in S305), the angular velocity determination unit 11 determines whether the angular velocity storage value in the Y-axis direction is “+” or not (S311). If the angular velocity storage value in the Y-axis direction is “+” (Yes in S311), the angular velocity determination unit 11 determines whether the angular velocity storage value in the X-axis direction is “−” or not (S312). If the angular velocity storage value in the X-axis direction is “−” (Yes in S313), the angular velocity determination unit 11 determines that the left upper arm is the measurement region (S313). In this regard, a method of determining a measurement region is the same as described in steps S307 and S309, and thus a specific description will be omitted hereinafter.

On the other hand, if the angular velocity storage value in the X-axis direction is not “−” (No in S312), the angular velocity determination unit 11 determines whether the angular velocity storage value in the X-axis direction is “+” or not (S314). If the angular velocity storage value in the X-axis direction is “+” (Yes in S314), the angular velocity determination unit 11 determines that the right upper arm is the measurement region (S315).

Also, in step S303, if the acceleration in the X-axis direction is not 0 (No in S303), the angular velocity determination unit 11 obtains the angular velocity storage value, which has been detected in step S301 and stored in the memory 102 (S317). Next, the angular velocity determination unit 11 determines whether the angular velocity storage value in the Z-axis direction is “+” or not (S318). If the angular velocity storage value in the Z-axis direction is “+” (Yes in S318), the angular velocity determination unit 11 determines whether the angular velocity storage value in the Y-axis direction is “0” or not (S319). If the angular velocity storage value in the Y-axis direction is “0” (Yes in S319), the angular velocity determination unit 11 determines that the left femoral region is the measurement region (S320).

On the other hand, if the angular velocity storage value in the Y-axis direction is not “0” (No in S319), the angular velocity determination unit 11 determines whether the angular velocity storage value in the Y-axis direction is “+” or not (S321). If the angular velocity storage value in the Y-axis direction is “+” (Yes in S321), the angular velocity determination unit 11 determines that the right femoral region is the measurement region (S322).

Also, in step S318, if the angular velocity storage value in the Z-axis direction is not “+” (No in S318), the angular velocity determination unit 11 determines whether the angular velocity storage value in the Z-axis direction is “−” or not (S324). If the angular velocity storage value in the Z-axis direction is “−” (Yes in S324), the angular velocity determination unit 11 determines whether the angular velocity storage value in the Y-axis direction is “+” or not (S325). If the angular velocity storage value in the Y-axis direction is “+” (Yes in S325), the angular velocity determination unit 11 determines that the left femoral region is the measurement region (S326). On the other hand, if the angular velocity storage value in the Y-axis direction is not “+” (No in S325), the angular velocity determination unit 11 determines whether the angular velocity storage value in the Y-axis direction is “0” or not (S327). If the angular velocity storage value in the Y-axis direction is “0” (Yes in S327), the angular velocity determination unit 11 determines that the right femoral region is the measurement region (S328).

Also, in step S324, if the angular velocity storage value in the Z-axis direction is not “−” (No in S324), the angular velocity determination unit 11 determines whether the angular velocity storage value in the Z-axis direction is “0” or not (S330). If the angular velocity storage value in the Z-axis direction is “0” (Yes in S330), the angular velocity determination unit 11 determines whether the angular velocity storage value in the X-axis direction is “−” or not (S331). If the angular velocity storage value in the X-axis direction is “−” (Yes in S331), the angular velocity determination unit 11 determines that the left abdominal region is the measurement region (S332). On the other hand, if the angular velocity storage value in the X-axis direction is not “−” (No in S331), the angular velocity determination unit 11 determines whether the angular velocity storage value in the X-axis direction is “+” or not (S333). If the angular velocity storage value in the X-axis direction is “+” (Yes in S333), the angular velocity determination unit 11 determines that the right abdominal region is the measurement region (S334).

In this regard, as is apparent from FIG. 12, the processing in FIG. 13 is repeated every time the measurement region is changed. Accordingly, in the second embodiment, the position of the mobile terminal 10 is returned to the reference state once, and then is moved to a measurement region every time the measurement region is changed.

As described above, by the second embodiment, it is possible to improve the degree of freedom in the measuring order of the measurement region. That is to say, it is possible for the user to freely select a first measurement region.

In this regard, in FIG. 11, there is no duplication in combinations of the angular velocity detection values in the X-axis direction, in the Y-axis direction, and in the Z-axis direction. Accordingly, the measurement region may be determined on the basis of the angular velocities detected in the process of the movement of the mobile terminal 10 from the reference state to the measurement region without using the acceleration detection values. However, it is possible to improve determination precision of a measurement region using acceleration detected in a stable state of the posture of the mobile terminal 10.

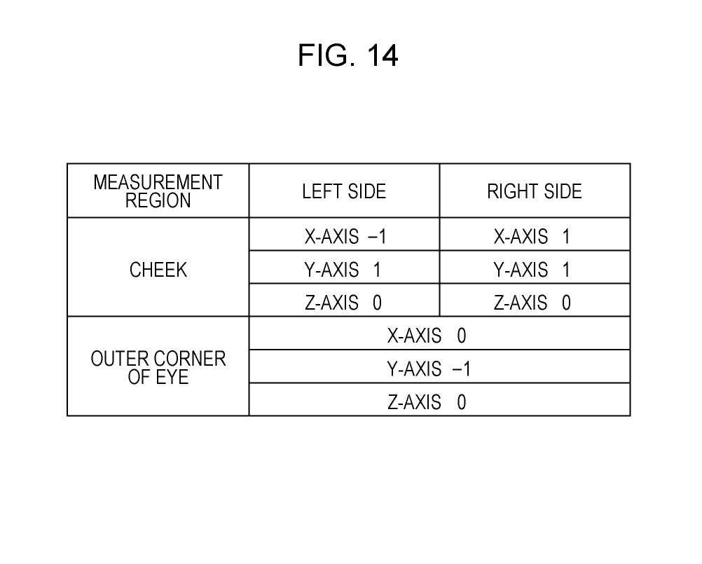

In this regard, each of the above-described embodiments may be applied to a measuring apparatus that is targeted for measuring things other than a subcutaneous fat thickness. For example, if the measurement target is skin moisture or skin oil, the measurement regions are assumed to be a part of a face, such as a cheek, an outer corner of an eye, and so on. In the case where skin moisture or skin oil is measured using a mobile terminal 10 according to the present embodiment, in order to automatically determine a measurement region on the basis of the detection values by the acceleration sensor 107, it is possible to use information as illustrated in FIG. 14.

FIG. 14 is a diagram illustrating a relationship between detection values of an acceleration sensor and a measurement region of a part of a face. FIG. 14 illustrates an example of detection values by the acceleration sensor 107 in the case where left and right cheeks, and left and right outer corner of eyes are the measurement regions. If the user wants to determine an upper arm, an abdominal region, a femoral region, and so on to be the measurement regions of skin moisture or skin oil, it is possible to use the information illustrated in FIG. 8.

In this manner, if the detection values of the acceleration sensor 107 at the time of measuring a region of interest for each region, and the detection values of the angular velocity sensor 106 at the time of measuring a region of interest for each region, and so on are measured in advance, it is possible to apply the present embodiment to various kinds of measuring apparatuses.

Also, in the present embodiment, examples have been given of the case where a user measures a part of the body of himself or herself. The present embodiment may be applied to the case where a user measures the other person, and the cases of measuring some numbers, an amount of something, a weight, or a length, and so on of each part of an object.

Also, the reference state may not be a state in which the front face of the mobile terminal 10 is not exactly facing the face of the user. The reference state may be suitably determined in view of the state of an apparatus used for measuring at the time of operation input, and so on.

In this regard, in the present embodiment, the mobile terminal 10 is an example of a measuring apparatus. The angular velocity sensor 106 is an example of the first detection unit. The acceleration sensor 107 is an example of the second detection unit. The angular velocity determination unit 11 and the acceleration determination unit 12 are examples of the determination unit. The reference state is an example of a predetermined position.

In the above, the detailed descriptions have been given of the embodiments of the present disclosure. However, the present disclosure is not limited to a specific embodiment. It is possible to make various alternations and modifications within the spirit and scope of the appended claims.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A measuring apparatus for measuring predetermined information on a plurality of regions to be measured, the measuring apparatus comprising:
a first detection unit configured to detect a rotation mode of the measuring apparatus; and
a determination unit configured to determine a region measured by the measuring apparatus on the basis of the rotation mode detected by the first detection unit in a movement process of the measuring apparatus from a predetermined position to any one of the regions.

2. The measuring apparatus according to claim 1, further comprising:
a second detection unit configured to detect a posture of the measuring apparatus,
wherein the determination unit is configured to determine a region measured by the measuring apparatus on the basis of the rotation mode detected by the first detection unit in a movement process of the measuring apparatus from a predetermined position to any one of the regions, and the posture detected by the second detection unit after the movement to any one of the regions.

3. The measuring apparatus according to claim 2,
wherein the determination unit is configured to determine whether a holding hand of the measuring apparatus is a right hand or a left hand on the basis of the rotation mode detected by the first detection unit in the movement process, and to determine the region measured by the measuring apparatus on the basis of the determination result, and the posture detected by the second detection unit after the movement.

4. A method of measuring a region, the method causing a measuring apparatus configured to measure predetermined information on a plurality of regions to be measured to perform processing comprising:
detecting a rotation mode of the measuring apparatus in a movement process of the measuring apparatus from a predetermined position to any one of the regions; and
determining a region measured by the measuring apparatus on the basis of the detected rotation mode.

5. A program for causing a measuring apparatus configured to measure predetermined information on a plurality of regions to be measured to perform processing comprising:
detecting a rotation mode of the measuring apparatus in a movement process of the measuring apparatus from a predetermined position to any one of the regions; and
determining a region measured by the measuring apparatus on the basis of the detected rotation mode.
